(19) United States
(12) Patent Application Publication
(10) Pub. No.: US 2010/0021525 A1
(43) Pub. Date: Jan. 28, 2010

(54) Method for Producing Sterol Formulations

(75) Inventors: Peter Horlacher, Bellenberg (DE); Dieter Hietsch, Illterissen (DE)

Correspondence Address: FOX ROTHSCHILD LLP, 2000 MARKET STREET, PHILADELPHIA, PA, 19103, US

(73) Assignee: Cognis IP Management GmbH, Duseldorf (DE)

(21) Appl. No.: 12/517,599

(22) PCT Filed: Nov. 24, 2007
(86) PCT No.: PCT/EP07/10230

§ 371 Date: Jun. 4, 2009

(30) Foreign Application Priority Data
Dec. 4, 2006 (EP) EP06025004.0

(51) Int. Cl. A61K 9/14; A23L 1/30

(52) U.S. Cl. 424/439; 424/491

(57) ABSTRACT

The invention relates to a method for producing coated sterol powders. According to said method, a) micronized sterol and/or stanol particles are provided in a mixer, b) the micronized sterol and/or stanol particles are wetted with a proteinaceous aqueous solution/dispersion, c) the wetted particles are mixed well and then dried, and d) the dried mixture is optionally size-reduced in a mill. Owing to their good wettability, the sterol-containing formulations produced according to this method can be incorporated into food items without technical complications and have good organoleptic and sensory properties especially when used in drinks and dairy products.

### FIELD OF THE INVENTION

The invention is in the field of foods and relates to a method of producing readily wettable phytosterol-containing formulations, the preparations produced according to this method, and also products, in particular foods, which contain these formulations.

### PRIOR ART

Numerous possible methods of formulation are known from application technology for being able to incorporate slightly water-soluble phytosterols and phytostanols which are used as cholesterol-lowering agents into food preparations or pharmaceutical products.

Numerous patent applications describe how the availability of sterols can be improved via reducing particle sizes, principally by micronization. For instance, German laid-open application DE 102 53 111 A1 describes pulverulent phytosterol formulations having a median particle size of 0.01 to 100 μm which may be readily redispersed in water. Preferably, use is made of hydrophilic auxiliaries as protective colloids. For producing the powders, use is made of organic solvents to the disadvantage of ecology and acceptability. International application WO 2005/074717 A1 also uses a type of protective colloid by embedding sterols into a matrix which contains proteins and carbohydrates. The total sterol content in the formulation, however, is small, owing to the high fraction of auxiliaries.

A further method for producing a sterol dispersion in which the particle size distribution of the sterols is from 0.1 to 30 μm, may be found in the international applications WO 03/105611 and WO 2005/049037. As in this method, frequently micronization of the sterol particles alone is insufficient in order to achieve good incorporability. Although the bioavailability of the finely dispersed particles may be improved by increasing the surface area, especially the micronized particles are poorly wettable, aggregate readily and generally float on aqueous surfaces. A disadvantage is in addition the thermal stress of the dispersions during production.

Frequently, the ground sterol can only be dispersed in a drink using special methods, for which intense mixing is necessary. However, these apparatuses are not usually available to the end user, the food manufacturer.

Therefore, many manufacturers combine micronization of sterols with the additional use of emulsifiers. An example thereof are the preparations claimed in European patent EP 0897671 B1 having sterols and sterol esters having a particle size of a maximum of 15 μm in a mixture with selected emulsifiers, wherein the weight ratio of emulsifier to sterol in the aqueous phase is less than 1:2.

In the international patent application WO 03/086468 A1, pulverulent sterol ester formulations having a low protein content and mono- and diglycerides as emulsifiers are disclosed. Even if these are distinguished by good acceptability and have already been known as food emulsifiers over a long time period, attempts are made to decrease the amount of the emulsifiers, or even to avoid them completely, since emulsifiers also affect the bioavailability of other substances present in the foods or can adversely affect the stability of the formulations.

Other methods of improving the solubility and dispersibility, such as formulation as emulsions, microemulsions, dispersions, suspensions or complexing with cyclodextrins or bile salts are presented in international patent application WO 99/63841 A1. Proposed supports are PEG, PVP, copolymers, cellulose ethers and cellulose esters. A disadvantage of these formulations is an often very high fraction of excipients which are added to the final formulations in order to achieve a sufficient concentration of sterols. Also, direct use of food base materials as supports for pulverized sterols in the form of a premix is disclosed by EP 1 003 388 B1. The selection of proteins as support substances for non-esterified sterols and stanols is disclosed in WO 01/37681.

In particular, processing non-esterified sterols and stanols which are still very much more hydrophobic than their esterified derivatives makes high demands on the production method. Free ground sterols in addition, have the disadvantage that they have a low minimum ignition energy (MIE <3 mJ), and therefore these products are categorized as extremely sensitive to ignition. Therefore, when free sterols are used, corresponding safety precautions must be heeded.

A possible method of producing sterol-containing microparticles can be found in European patent EP 1148793 B1. It is based on high-energy homogenization. However, a powder produced thereby based on aqueous suspension media has an inadequate homogeneity and can only be redispersed with difficulty. A disadvantage of many sterol-containing powder formulations is the agglomeration behavior of the free sterols on storage. During storage, especially if they are stored under pressure, severe clumping or lump formation is observed, and the solid uncontrolled agglomerates must again be comminuted in order then to be able to be processed.

In the international patent application WO 2006/020980 A1, agglomerates of sterol particles are described. The production method is a size-enlargement granulation of adhesive granules in which the micronized sterol particles are wetted with a suspension medium in which a binder is in part or completely dissolved. The suspension medium is removed after the wetting, in such a manner that the remaining agglomerates have a size of 150 to 850 μm. This method requires a high use of apparatus and must be controlled very precisely, in order that the agglomerates produced have the desired stability.

It was an object of the present invention to provide highly concentrated sterol-containing formulations, which may be produced using simple and rapid processes, and enable good and rapid dispersion and incorporation of non-esterified sterols and/or stanols in foods, wherein the formulations should have good sensory and organoleptic properties in the foods.

### DESCRIPTION OF THE INVENTION

The invention relates to methods for producing coated sterol powders in which

a) micronized sterol particles and/or stanol particles are charged into a mixer,
b) the micronized sterol and/or stanol particles are moistened with a proteinaceous aqueous solution/dispersion,
c) the moistened particles are mixed well and subsequently dried, and
d) if appropriate the dried material is comminuted in a mill.

If the proteinaceous aqueous solution is merely sprayed on during mixing of the micronized powder, surprisingly, despite use of a simple ploughshare mixer, after the drying (step c), a subsequent milling of the powder (step d) can be omitted, without the particle size of the coated sterol powder deviating substantially from the size of the material originally used. Only a slight particle size enlargement can be detected.

Even after carrying out step d) the particle size distribution should be slightly larger than that of the micronized sterols and/or stanols used.

Thus the sterol particles and/or stanol particles used in step a) have a particle size distribution having a D_{90%} of a maximum of 100 μm, preferably a maximum of 40 μm, and particularly preferably a maximum of 30 μm. The particle size distribution was measured using an instrument from Beckman Coulter, type LS 230, and calculated as a volume distribution. The measurement is performed in an aqueous suspension.

After coating by spraying, loose granules are formed which if appropriate can be classified in such a manner that the powder has a particle size distribution having a D_{90%} of a maximum of 1000 μm. If the coating proceeds via pasting, generally milling of the dried material follows, with which the particle size can then be adjusted to the desired extent.

The use of proteinaceous aqueous coating materials and particularly of milk powder or sodium caseinate as coating material leads to an improved water dispersability compared with sterol formulations having other hydrophilizing auxiliaries as are already described in the international patent application WO 2006/020980 A1.

The method according to the invention enables powders also to be produced with free non-esterified sterols or stanols which enable easy further processing of the lipophilic active ingredients in foods, in particular drinks. The powder has a low agglomeration tendency and therefore good flow properties. It is distinguished by good homogeneity and, owing to its improved wettability, can be further processed without great technical resources, wherein a homogeneous distribution in the final formulation is also rapidly achieved. Owing to the coating of the sterol surface with the hydrophilic additives, the organoleptic properties and the sensory properties are decisively improved. The coated powder does not stick to teeth and oral mucosa, and therefore the unpleasant sterol taste which leads to considerable taste impairments in the foods which contain active ingredients is completely suppressed. In this case the fraction of the auxiliaries can nevertheless be kept low, and therefore a highly concentrated sterol and/or stanol powder is obtained by which during further processing a sufficient amount of sterols and stanols can be achieved in the food or another final formulation without a high input of powder.

By using hydrophilizing proteinaceous auxiliaries, not only are solubilization properties and dispersion properties improved, but surprisingly these powders also exhibit an increased storage stability compared with pure milled sterols which have a high agglomeration tendency.

The method allows organic solvents or heating the formulation to be avoided in the processing of non-esterified sterols and stanols, and despite the aqueous medium, allows the omission of emulsifiers having a high surface activity, especially of the type of lecithins, monoglycerides, diglycerides, polysorbates, sodium stearyl lactylate, glycerol monostearate, lactic acid esters and polyglycerol esters. The low emulsifying properties of the hydrophilizing auxiliaries, in particular of the caseinates and the milk powder are sufficient in order to ensure the homogeneity of the powder which is produced and ready redispersibility and processability. The omission of further emulsifiers simplifies the further processing by reducing possible incompatibilities with other food constituents and decreases the occurrence of incompatibilities with the consumer.

Surprisingly, other auxiliaries such as glucose and gum arabic, despite their strongly hydrophilic properties, do not have such good dispersion properties as skimmed milk powder or sodium caseinate. This behavior may possibly be explained by the fact that gum arabic and glucose dissolve too rapidly in water and as a result the coating of the sterols is partially detached, such that the lipophilic character of the sterol particle surface is again increasingly expressed.

The resultant powders are distinguished by improved free-flowing properties, better stirability into water and a higher bulk density compared with customary finely ground sterols. The coated powders can be added by simple stirring into aqueous systems, such as cold water, juices or milk.

The production may be implemented with a very low use of apparatus. For instance, the mixing process can be carried out using conventional commercial powder mixers. Simple blade mixers, kneading mixers or ploughshare mixers are sufficient for an effective mixing operation. This operation should be completed in one to a maximum of two hours.

The micronized sterol and/or stanol powder can then be moistened by simple spraying with the proteinaceous aqueous solution or by direct addition of the solution into the mixer (pasting). Conventional coating methods such as, for example, fluidized-bed methods or extrusion methods, are not excluded, but owing to the structure of the apparatus, simple stirrers with a spraying device are preferred.

For the drying (step c), conventional drying methods such as vacuum drying or spray drying are suitable, here also vacuum drying is sufficient in order to obtain a fine free-flowing powder.

If, for the moistening, pasting with the proteinaceous solution is selected, then, depending on agglomeration and particle size distribution after the vacuum drying, a subsequent milling operation is necessary in order to obtain the desired particle size distribution. The mill to be used in step d) for grinding should be based on a rotor-stator principle or be a ball mill. The use of resources of the production apparatus is then also still very low. Cooling of the milling material can be omitted owing to the very low heating of the formulation.

The powder resulting after drying has a very high sterol content of at least 90% by weight, preferably at least 93% by weight, and particularly preferably at least 95% by weight, based on the total weight of the coated powder.

Therefore, a further subject matter is pulverulent coated sterol preparations comprising particles which contain a core of sterols and/or stanols and also, if appropriate, further lipophilic auxiliaries and a coating of sodium caseinate powder and/or milk powder, and also, if appropriate, further hydrophilic auxiliaries, with the proviso that the sterol preparation contains at least 90% by weight, preferably at least 93% by weight, and particularly preferably at least 95% by weight of sterols and/or stanols based on the total formulation. Preferably, the coated formulations are free from emulsifiers having a high surface activity, in particular those which are selected from the group formed by lecithins, monoglycerides, diglycerides, polysorbates, sodium stearyl lactylate, glycerol monostearate, lactic acid esters and polyglycerol esters.

The sterol-containing formulations produced by this method can be incorporated in a simple manner into foods, in particular into milk, milk drinks, whey drinks, yoghurt drinks, margarine, fruit juices, fruit juice mixtures, fruit juice drinks, vegetable juices, carbonated and non-carbonated drinks, soymilk drinks or protein-rich liquid food replacement drinks, and also fermented milk preparations, yoghurt, drinking yoghurt or cheese preparations, but also into pharmaceutical preparations.

The invention further relates to food preparations which contain sterol/stanol formulations of said composition. They are used preferably in drinks and milk products which then contain 0.1 to 50% by weight, preferably 1 to 20% by weight, of the pulverulent coated preparations based on the total weight of the foods.

Sterol and/or Stanol

In the present invention, sterols obtained from plants and plant raw materials, termed phytosterols and phytostanols, are used. Known examples are ergosterol, brassicasterol, campesterol, avenasterol, desmosterol, clionasterol, stigmasterol, poriferasterol, chalinosterol, sitosterol and mixtures thereof, among these, use is preferably made of β-sitosterol and campesterol. Likewise, the hydrogenated saturated forms of the sterols, termed stanols, come under the compounds used, and here also β-sitostanol and campestanol are preferred. As plant raw material sources, there serve, inter alia, seeds and oils of soybeans, canola, palm kernels, corn, coconut, rape, sugarcane, sunflower, olive, cotton, soybean, peanut or products from tall oil production.

Proteinaceous Auxiliaries and/or Proteins

Proteinaceous auxiliaries and proteins used are preferably milk powder and/or whey powder and/or casein and/or caseinates. Milk powders such as commercially obtainable whole milk and skimmed milk powders which have been obtained from the respective milk quality grades by drying are particularly suitable. They can be used in mixtures with other proteins or as sole support. If other proteins are added or proteins are used instead of milk powder as support, these are taken to include isolated proteins which are obtained from natural animal and plant sources and are added during production of the pulverulent preparations. Possible sources of proteins are plants such as wheat, soybean, lupin, corn or sources of animal origin such as eggs or milk.

Skimmed milk powder, in the context of the present invention, is particularly preferred since it has sufficient hydrophilizing properties without therefore also simultaneously exhibiting the disadvantages of such food emulsifiers described at the outset, which are otherwise customarily used especially for producing drinks and milk products, especially fermentation products such as yoghurt. In addition, skimmed milk powder best masks the typical unpleasant sterol flavor and formulations having this additive have improved sensory properties compared with other auxiliaries. It has been found that sterol formulations coated with skimmed milk powder and sodium caseinate have particularly good dispersion properties, since they firstly have sufficiently good hydrophilicity in order to increase the wettability in aqueous systems and secondly they do not have excessively good water solubility to be dissolved directly from the sterol surface. An amount of a maximum of 10% by weight, preferably a maximum of 7% by weight, and particularly preferably a maximum of 5% by weight, based on the total weight of the coated powder, is sufficient in order to achieve the improved dispersion and processing properties. As a result of the fact that the micronized sterol is only coated and is not embedded in a relatively large amount of hydrophilizing auxiliaries, as described in international application WO 2005/074717 A1, the total content of sterols in the formulation can be kept very high.

### Further Auxiliaries

As further auxiliaries, the preparations according to the invention can contain antioxidants, preservatives and flow enhancers. Examples of possible antioxidants or preservatives are tocopherols, lecithins, ascorbic acid, parabens, butylated hydroxytoluene or butylated hydroxyanisole, sorbic acid or benzoic acid and salts thereof. Preferably, tocopherols are used as antioxidants.

As flow regulator and improver, silicon dioxide can be used.

### EXAMPLES

### Example 1

### Coating by Spraying and Drying

450 g of micronized sterol powder (Vegapure® FTE, Cognis, Germany, of particle size <100 μm) were charged into a laboratory mixer (Lödige, type M5R) at room temperature. A solution of 50 g of skimmed milk powder (spray-dried skimmed milk powder ADPI grade, supplier: Almil, Bad Homburg) in 300 g of water (60° C.) was sprayed on with mixing (speed of rotation 50%) (spraying time 5 min). Mixing is continued for 30 min at 30-40° C. (speed of rotation 15%). Subsequently the mixture was dried in a vacuum at 60°/1 mbar to a water content <5%.

This produced fine granules having improved free-flowing properties, better stirability into water and a higher bulk density compared with the sterol powder used.

### Example 2

### Coating by Spraying and Drying

450 g of sterol powder (Vegapure FTE, Cognis Germany, particle size <100 μm) are charged into a laboratory mixer (Lödige, type M5R—ploughshare mixer) at room temperature. A solution of 50 g of skimmed milk powder (spray-dried skimmed milk powder ADPI grade, supplier: Almil, Bad Homburg) in 250 g water (60° C.) was sprayed on with mixing (speed of rotation 50%) (spraying time 5 min). Subsequently mixing was continued for a further 30 min at 30-40° C. (speed of rotation 15%).

Thereafter the mixture was dried in a vacuum to a water content <5% (simulation of a reactor drying by a rotary evaporator/bath temperature 70° C./pressure: 40 mbar/time 2 h).

This likewise produced fine granules having improved free-flowing properties, water wettability and a higher bulk density.

### Example 3

### Coating by Pasting, Drying and Grinding

a) 7% by weight of skimmed milk powder based on the final formulation (hydrophilized sterol powder)

85 g of skimmed milk powder (spray-dried skimmed milk powder ADPI grade, supplier: Almil, Bad Homburg) were dissolved in 1125 g of deionized water at 80° C. In a kneader (kitchen appliance from Braun), 1125 g of micronized sterol (Vegapure® FTE) were charged and the milk powder solution was added. This produced a smooth dough which was dried in a vacuum drying cabinet (50° C./<50 mbar) and subsequently milled in a cutting mill (Retsch Grindomix GN 200; conditions: 2000 rpm, 30 seconds).

Similarly to example 3, the following hydrophilic coatings were applied:

3 b) 5% by weight of skimmed milk powder
3 c) 10% by weight of skimmed milk powder
3 d) 10% by weight of Na-caseinate (from Meggle, Emulac Na)
3 e) 5% by weight of dried glucose syrup (from Roquette, Glucidex IT 33)
3 f) 5% by weight of gum arabic (Alfred L. Wolff, Gum Arabic type 8074)

All the hydrophilized sterol powders produced according to example 3 were distinguished by good free-flowing properties, pourability, and flowability and good storage capacity without lump formation. With respect to water dispersibility, however, the various coated sterol powders showed great differences:

The sterol powders produced according to example 3 were dispersed in water at room temperature. For this, approximately 250 ml of the liquid under test were placed in a glass beaker and stirred at 100 rpm. 2.5 g of the respective powder were added to the stirred liquid and the dispersion behavior was assessed.

TABLE 1
Water dispersability of sterol powders coated in different ways

| Hydrophilic coating | 5% by weight | 10% by weight |
|---|---|---|
| Skimmed milk powder | Good | Very good |
| Sodium caseinate | — | Very good |
| Glucose | Poor | — |
| Gum arabic | Poor | — |

Surprisingly, glucose and gum arabic, despite their highly hydrophilic properties, did not have dispersion properties as good as skimmed milk powder or sodium caseinate. Large amounts of these sterol particles which were coated with glucose and gum arabic floated and remained on the stirred water surface.

What is claimed is:

1. A method of producing coated sterol powders comprising:
a) charging micronized sterol particles and/or stanol particles into a mixer,
b) moistening said micronized sterol and/or stanol particles with an aqueous solution/dispersion of a protein or proteinaceous auxiliary,
c) thoroughly mixing the moistened particles and subsequently drying, and
d) optionally comminuting the dried material in a mill.

2. The method of producing coated sterol powders of claim 1, wherein said protein or proteinaceous auxiliary is selected from milk powder and/or caseinates.

3. The method of producing coated sterol powders of claim 1 and/or 2, wherein said micronized sterol and/or stanol particles are sprayed with said aqueous solution/dispersion in such a manner that, after drying of the particles, no comminution (step d) is required.

4. The method of producing coated sterol powders of claim 1, wherein the particle size distribution, D_{90%}, of the coated powder after carrying out step d), is greater than the D_{90%} of said micronized sterol particles and/or stanol particles used in step a).

5. The method of producing coated sterol powders of claim 1, wherein sterol particles and/or stanol particles of a particle size distribution having a D_{90%} of a maximum of 50 μm are used in step a) and, after step d) is carried out, the particle size distribution of said coated sterol powder has a D_{90%} of a maximum of 1000 μm.

6. The method of producing coated sterol powders of claim 1, wherein a ploughshare mixer is used in step a).

7. A pulverulent coated sterol preparation comprising particles which contain a core of sterols and/or stanols and optionally, lipophilic auxiliaries, and a coating of sodium caseinate powder and/or milk powder and, optionally, additional hydrophilic auxiliaries, provided that said coated sterol preparation contains at least 90% by weight of sterols and/or stanols, based on the preparation.

8. A food containing 0.1 to 50% by weight of the pulverulent coated sterol preparation as claimed in claim 7.

9. A drink or milk product containing 0.1 to 50% by weight of the pulverulent coated sterol preparation as claimed in claim 7.
